(19) United States
(12) Patent Application Publication
(10) Pub. No.: US 2023/0271018 A1
(43) Pub. Date: Aug. 31, 2023

(54) NON-TRANSITORY COMPUTER-READABLE MEDIUM, PORTABLE INFORMATION TERMINAL, SYSTEM, AND METHOD FOR NOTIFICATION ABOUT ACTIVATION OF AUTOMATED EXTERNAL DEFIBRILLATOR

(71) Applicant: Nihon Kohden Corporation, Tokyo (JP)

(72) Inventors: Tsutomu Wakabayashi, Saitama (JP); Yuji Igawa, Saitama (JP); Kanchi Ito, Saitama (JP)

(73) Assignee: Nihon Kohden Corporation, Tokyo (JP)

(21) Appl. No.: 18/008,129

(22) PCT Filed: May 28, 2021
(86) PCT No.: PCT/JP2021/020421

§ 371 Date: Dec. 2, 2022

(30) Foreign Application Priority Data
Jun. 5, 2020 (JP) 2020-098726

(51) Int. Cl. A61N 1/372; A61N 1/39

(52) U.S. Cl. CPC A61N 1/37282; A61N 1/3904; A61N 1/3937

(57) ABSTRACT

A non-transitory computer-readable medium has recorded thereon a program that makes circuitry of a portable information terminal execute: a detection step of detecting presence/absence of an automated external defibrillator in an activated state based on presence/absence of a wireless signal sent from the automated external defibrillator, the wireless signal becoming detectable in response to activation of the automated external defibrillator having a wireless communication module; and a first transmission step of transmitting notification information for notification about the activation of the automated external defibrillator to a management computer in response to the automated external defibrillator in the activated state being detected in the detection step.

### TECHNICAL FIELD

The present disclosure relates to a program, a portable information terminal, a system, and a method for notification about activation of an automated external defibrillator.

### BACKGROUND ART

When an automated external defibrillator (hereinafter also referred to as “AED”) has been used for rescue, for example, a user of the AED may want to contact an administrator managing the AED, an emergency medical team, or the like (hereinafter also referred to as “administrator or the like”) by phone or the like to notify the administrator or the like of the fact that the rescue using the AED has been performed.

For example, Patent Literature 1 discloses a system in which an activated AED serves as a main actor to make communication connection with a mobile terminal owned by a user to automatically start software on the mobile terminal so as to prompt the user to make a phone call to an EMS (Emergency Medical Services) on a screen of the mobile terminal.

### CITATION LIST

### Patent Literature

PTL 1: JP-T-2020-503920

### SUMMARY OF INVENTION

### Technical Problem

In the system disclosed in the Patent Literature 1, the activated AED, i.e. the AED in use for rescue activity detects the connectable mobile terminal and makes communication connection with the mobile terminal. That is, there is still room for reducing a load on the AED In addition, the user himself/herself sends notification to the EMS. Therefore, there is also room for an improvement for sending the notification more surely.

An object of the present disclosure is to provide a program that can more surely contact an administrator or the like about a fact that an automated external defibrillator has been activated even without making communication connection with the activated automated external defibrillator, a portable information terminal, a system, and a method for notification about the activation of the automated external defibrillator.

### Solution to Problem

According to a first aspect of the present disclosure, there is provided a program executed in a portable information terminal including a processor so that the program makes the processor execute:

a detection step of detecting presence/absence of an automated external defibrillator in an activated state based on presence/absence of a wireless signal sent from the automated external defibrillator, which wireless signal can be detected according to activation of the automated external defibrillator having a wireless communication module; and a first transmission step of transmitting notification information for notification about the activation of the automated external defibrillator to a management computer when the automated external defibrillator in the activated state has been detected.

According to a second aspect of the present disclosure, there is provided a portable information terminal including:
a detector that detects presence/absence of an automated external defibrillator in an activated state based on presence/absence of a wireless signal sent from the automated external defibrillator, which wireless signal can be detected according to activation of the automated external defibrillator having a wireless communication module; and
a first transmitter that transmits notification information for notification about the activation of the automated external defibrillator to a management computer when the automated external defibrillator in the activated state has been detected.

According to a third aspect of the present disclosure, there is provided a system that is realized by one or more portable information terminals, and a management computer capable of making communication connection with the portable information terminals, wherein:
each of the portable information terminals includes
   a detector that detects presence/absence of an automated external defibrillator in an activated state based on presence/absence of a wireless signal sent from the automated external defibrillator, which wireless signal can be detected according to activation of the automated external defibrillator having a wireless communication module, and
   a first transmitter that transmits notification information for notification about the activation of the automated external defibrillator to the management computer when the automated external defibrillator in the activated state has been detected; and
the management computer includes
   a receiver that receives the notification information transmitted by the first transmitter, and
   a third transmitter that transmits predetermined information to an arbitrary computer apparatus when the notification information has been received.

According to a fourth aspect of the present disclosure, there is provided a method for notification about activation of an automated external defibrillator, which is executed in a portable information terminal including a processor so that the method makes the processor execute:
a detection step of detecting presence/absence of the automated external defibrillator in an activated state based on presence/absence of a wireless signal sent from the automated external defibrillator, which wireless signal can be detected according to activation of the automated external defibrillator having a wireless communication module; and
a first transmission step of transmitting notification information for notification about the activation of the automated external defibrillator to a management computer when the automated external defibrillator in the activated state has been detected.

### Advantageous Effects of Invention

According to the aforementioned configurations, it is possible to provide a program that can more surely contact an administrator or the like about a fact that an automated external defibrillator has been activated even without making communication connection with the activated automated external defibrillator, a portable information terminal, a system, and a method for notification about the activation of the automated external defibrillator.

### BRIEF DESCRIPTION OF DRAWINGS

FIG. 1 is a block diagram illustrating an example of a configuration of a system according to an embodiment of the present disclosure.

FIG. 2. is a conceptual diagram illustrating an action example of the system according to the embodiment of the present disclosure.

FIG. 3 is a flowchart for explaining an example of an action process executed in the system according to the embodiment of the present disclosure.

FIG. 4 is a flowchart for explaining an example of an action process executed in the system according to the embodiment of the present disclosure.

### DESCRIPTION OF EMBODIMENTS

An embodiment of the present disclosure will be described below by way of example with reference to the drawings. Identical or equivalent elements will be designated by the same reference signs or names correspondingly and respectively even in different drawings, and duplicate description thereof will be therefore omitted appropriately.

### Configuration of System

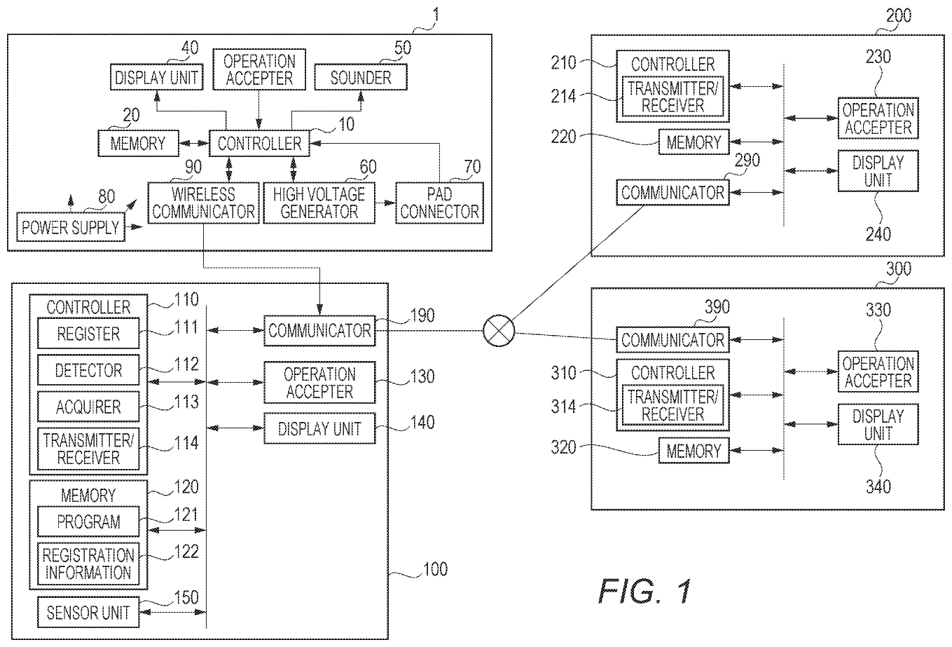

First, a configuration of a system 400 according to the embodiment of the present disclosure will be described using FIG. 1. FIG. 1 is a block diagram illustrating an example of the configuration of the system 400. The system 400 may include one or more automated external defibrillators (AEDs) 1, a portable information terminal 100, a management computer 200, and a computer apparatus 300.

In the system 400, the portable information terminal 100 detects, of the AEDs 1, an AED 1 in an activated state, and transmits notification information for notification about the activation of the AED 1 to the management computer 200. In addition, the management computer 200 transmits predetermined information to the computer apparatus 300 as occasion demands. The respective apparatuses that may be included in the system 400 and respective processors constituting the apparatuses will be described below in detail.

AED

Each of the AEDs 1 is provided with a controller 10, a memory 20, an operation accepter 30, a display unit 40, a sounder 50, a high voltage generator 60, a pad connector 70, a power supply 80, and a wireless communicator 90. The AED 1 may be a portable type AED or may be a stationary type AED. Incidentally, it is preferable that the AED 1 is not provided with a GPS sensor.

The controller 10 reads and executes a program etc. stored in the memory 20 to control various actions of the AED 1. The controller 10 has a processor such as a CPU (Central Processing Unit), a memory such as an ROM (Read Only Memory) or an RAM (Random Access Memory), an A/D converter, etc.

The controller 10 controls various actions for life rescue such as energy charging/discharging control, sequence control, A/D conversion, and electrocardiogram analysis. In addition, the controller 10 controls execution of a self-test of the AED 1. In the self-test, for example, the controller 10 checks states of a circuit for controlling the life rescue actions, the power supply 80, a defibrillation pad (not illustrated) connected to the pad connector 70, etc. to confirm whether they are normal or abnormal.

Further, the controller 10 may function as a wireless communication module. The controller 10, for example, performs transmission/reception of information to/from another apparatus such as the portable information terminal 100 through the wireless communicator 90.

At a predetermined timing such as after a series of the life rescue actions has been completed or after the self-test has been terminated, the controller 10 may transmit, to the portable information terminal 100 or another computer or the like, physiological information such as electrocardiogram data of a person in need for rescue, to whom the life rescue actions have been applied, or a result of the self-test.

The memory 20 stores a program necessary for the AED 1 to act, audio data, an adjustment value, the electrocardiogram data during the life rescue, the result of the self-test, etc. The memory 20 may include, for example, a secondary memory device such as a hard disk. A part of the memory 20 may be an external memory device that can be detachably attached to the AED 1.

The operation accepter 30 accepts an operation input from a user of the AED 1. The operation accepter 30 may include a power button for activating the AED 1, a check button for starting the self-test, a shock button for executing an electric shock, etc. Incidentally, for example, through the power button or the check button pressed down in a state in which the AED 1 is powered off or through a cover of the AED 1 turned into an open state, the AED 1 is brought into an activated state. The activated state means a state in which the AED 1 is capable of executing processings for life rescue (such as outputting of voice guidance, detection of attachment of the defibrillation pad, detection of an electrocardiogram, outputting of defibrillation shock energy, etc.).

The display unit 40 may include an indicator for displaying the result of the self-test, a liquid crystal display for displaying an instruction to the user of the AED 1 as a figure or characters, a display for displaying an electrocardiogram signal, etc. In addition, the display unit 40 may be provided with a touch panel or may function also as the operation accepter 30.

The sounder 50 issues various instructions to the user of the AED 1 by voice with reference to the audio data stored in the memory 20. In addition, when an abnormality has been found by the self-test, the sounder 50 emits a warning sound to notify the user of the abnormality.

The high voltage generator 60 carries out charging and discharging of energy used for defibrillation in accordance with a control signal from the controller 10. The pad connector 70 is connected to the defibrillation pad. The energy discharged by the high voltage generator 60 is transmitted to the person in need for rescue through the pad connector 70 and the defibrillation pad. In addition, the defibrillation pad fetches an electrocardiogram signal of the person in need for rescue. The electrocardiogram signal is, for example, filtered and amplified before being transmitted to the controller 10.

The power supply 80 includes a battery. The power supply 80 converts electric power supplied from the battery into a required voltage, and supplies the electric power to the aforementioned processors.

The wireless communicator 90 is an interface that enables wireless communication with another apparatus such as the portable information terminal 100. When the AED 1 is brought into the activated state, a wireless signal is sent from the wireless communicator 90 toward the outside. Here, the “wireless signal” means, for example, an audio signal such as a DTMF (Dual Tone Multi Frequency) signal, an optical signal such as an infrared beacon, or a radio signal such as a radio beacon. Incidentally, when the wireless signal is the audio signal, the sounder 50 may function as the wireless communicator 90.

It is preferable that, for example, first identification information by which the AED 1 can be identified individually is included in the wireless signal. In addition, it is preferable that the wireless signal is the radio signal. It is preferable that the wireless communicator 90 is compatible with a radio communication standard such as a wireless LAN (Local Area Network) standard or a Bluetooth (registered trademark) standard. In this case, the wireless communicator 90 forms a part of a wireless LAN module, a Bluetooth module, or the like. Thus, a network identifier such as an SSID (Service Set Identifier) by which the AED 1 can be identified individually, or the radio beacon including the first identification information such as an identifier of the AED 1 is sent as the wireless signal from the wireless communicator 90.

Portable Information Terminal

The portable information terminal 100 includes a controller 110, a memory 120, an operation accepter 130, a display unit 140, a sensor unit 150, and a communicator 190. These constituent elements are connected to one another through an internal bus. The portable information terminal 100 is not limited as long as it is a portable terminal apparatus. For example, the portable information terminal 100 may be a smartphone, a tablet or a wearable terminal.

The controller 110 includes a processor such as a CPU, and a memory such as an ROM or an RAM. The controller 110 controls various actions of the portable information terminal 100. The memory 120 stores a program or data required for the various actions of the portable information terminal 100. In the present embodiment, the memory 120 stores a program 121 that serves for detecting activation of an AED 1 to notify the management computer 200 of the activation of the AED 1, and registration information 122 that can be used in the program 121. The program 121 may be pre-installed in the portable information terminal 100 or may be distributed as an application program.

The operation accepter 130 accepts an operation input from a user of the portable information terminal 100. The operation accepter 130 may be constituted by a physical button, a touch panel, or the like. The display unit 140 may be constituted by a liquid crystal type or organic EL (Electro Luminescence) type display for displaying various information. In addition, the display unit 140 may be a touch panel or may function also as the operation accepter 130.

The sensor unit 150 includes a sensor for measuring position information of the portable information terminal 100. A GPS (Global Positioning System) sensor is listed as a specific example of such a sensor.

The communicator 190 is an interface that enables communication with another apparatus. The communicator 190 is an interface that can be compatible with a standard of the wireless signal sent from the AED 1. In addition, the communicator 190 enables communication with the management computer 200 through a communication network.

Various actions of the controller 110 will be described below in detail. The controller 110 reads and executes the program 121 to function as a registerer 111, a detector 112, an acquirer 113, and a transmitter/receiver 114.

The registerer 111 stores, as the registration information 122 in the memory 120, second identification information for individually identifying the AED 1 set as a detection target. The second identification information is information identical to or corresponding to the aforementioned first identification information. In addition, the registerer 111 may store, as the registration information 122 in the memory 120, individual information of the AED 1 set as the detection target, individual information of the portable information terminal 100, and individual information of the user of the portable information terminal 100. Here, the “individual information” means, for example, a model number, a terminal identification number or personal information of the user of the portable information terminal 100.

The detector 112 detects presence/absence of the AED 1 in the activated state, based on presence/absence of the aforementioned wireless signal that can be detected according to the fact that the AED 1 has been activated. Upon detection of the aforementioned wireless signal, the detector 112 determines that the AED 1 in the activated state is present.

When the wireless communicator 90 corresponds to the wireless LAN module, the detector 112 detects the SSID etc. included in the radio beacon sent from the wireless communicator 90. When the wireless communicator 90 corresponds to the Bluetooth module, the detector 112 detects the identifier etc. of the AED 1 included in the radio beacon sent from the wireless communicator 90. Moreover, when the wireless signal is an audio signal or an optical signal, the detector 112 detects presence/absence of a specific signal that has been set in advance.

The detector 112 can detect only the specific AED 1 by collating the detected first identification information with the second identification information registered in the registration information 122. It is preferable that a time interval at which the detector 112 executes a detection action can be set desirably by the user of the portable information terminal 100. The detector 112 detects presence/absence of the wireless signal at a predetermined time interval of, for example, thirty seconds, one minute, five minutes, ten minutes, or the like.

The acquirer 113 acquires the position information of the portable information terminal 100 when the AED 1 in the activated state has been detected by the detector 112. The position information is acquired by use of the GPS sensor or the like included in the sensor unit 150. In addition, the acquirer 113 may acquire the individual information stored in the registration information 122 when the AED 1 in the activated state has been detected by the detector 112.

The transmitter/receiver 114 performs transmission/reception of information to/from another apparatus. The transmitter/receiver 114 receives the aforementioned wireless signal from the AED 1 through the communicator 190. In addition, the transmitter/receiver 114 may receive physiological information such as electrocardiogram data of a person in need for rescue, or a result of a self-test.

In addition, the transmitter/receiver 114 transmits, to the management computer 200, notification information for notification about the activation of the AED 1 when the AED 1 in the activated state has been detected by the detector 112. The position information and/or the individual information which has been acquired by the acquirer 113 may be included in the notification information. In addition, the transmitter/receiver 114 may receive information from the management computer 200. The management computer 200 as a transmission destination may be set expressly through the operation accepter 130 of the portable information terminal 100.

Management Computer

The management computer 200 is provided with a controller 210, a memory 220, an operation accepter 230, a display unit 240, and a communicator 290. The constituent elements are connected to one another through an internal bus. The management computer 200 is a computer managing information transmitted from the one or more portable information terminals 100.

The controller 210 includes a processor such as a CPU, and a memory such as an ROM or an RAM. The controller 210 controls various actions of the management computer 200. The controller 210 functions also as a transmitter/receiver 214. The transmitter/receiver 214 performs transmission/reception of various information to/from the portable information terminal 100 and the computer apparatus 300. Specifically, the transmitter/receiver 214 receives the notification information (that may include the position information and/or the individual information) transmitted from the transmitter/receiver 114, the physiological information of the person in need for rescue, and information about the result of the self-test. In addition, the transmitter/receiver 214 transmits predetermined information to the computer apparatus 300 as occasion demands. For example, the physiological information of the person in need for rescue, or the position information received as the notification information may be included in the predetermined information.

The memory 220 stores a program or data required for the various actions of the management computer 200. The operation accepter 230 accepts an operation input from a user of the management computer 200. The display unit 240 may be constituted by a liquid crystal type or organic EL type display displaying various information. In addition, the display unit 240 may be a touch panel or may function also as the operation accepter 230. The communicator 290 is an interface that enables communication with another apparatus. The communicator 290 enables communication with the portable information terminal 100 and the computer apparatus 300 through the communication network.

Computer Apparatus

The computer apparatus 300 is provided with a controller 310, a memory 320, an operation accepter 330, a display unit 340, and a communicator 390. The constituent elements are connected to one another through an internal bus. The computer apparatus 300 is a computer that is, for example, installed in a medical site such as a hospital.

The controller 310 includes a processor such as a CPU, and a memory such as an ROM or an RAM. The controller 310 controls various actions of the computer apparatus 300. The controller 310 functions also as a transmitter/receiver 314. The transmitter/receiver 314 performs transmission/reception of various information to/from the management computer 200. Specifically, the transmitter/receiver 314 receives predetermined information transmitted from the transmitter/receiver 214. In addition, the transmitter/receiver 314 may transmit information to the management computer 200 as occasion demands.

The memory 320 stores a program or data required for the various actions of the computer apparatus 300. The operation accepter 330 accepts an operation input from a user of the computer apparatus 300. The display unit 340 may be constituted by a liquid crystal type or organic EL type display displaying various information. In addition, the display unit 340 may be a touch panel or may function also as the operation accepter 330. The communicator 390 is an interface that enables communication with another apparatus. The communicator 290 enables communication with the computer apparatus 300 through the communication network.

### Action Example of System

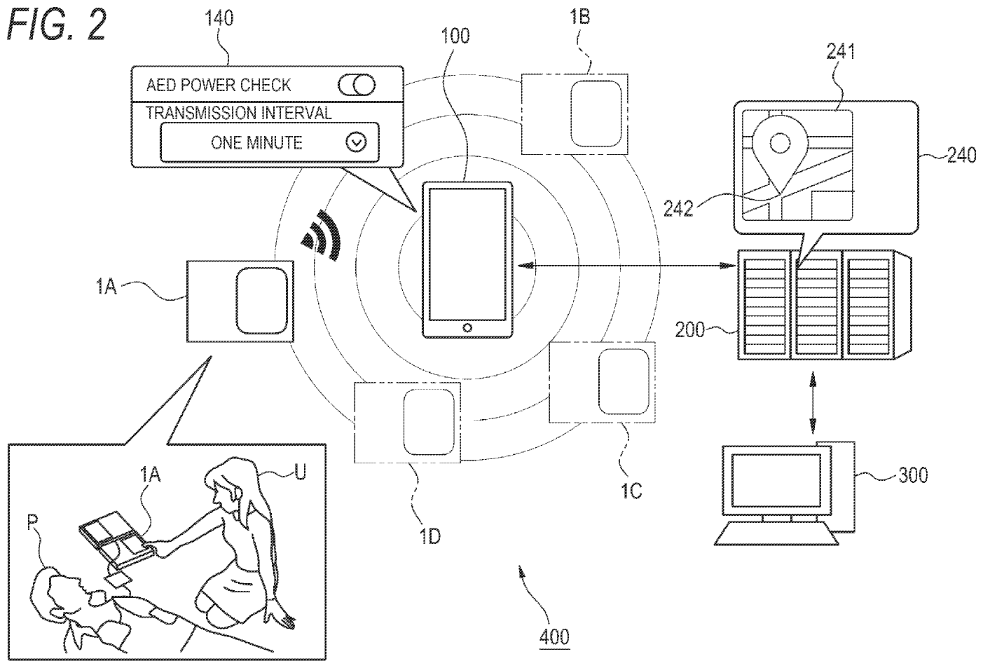

An action example of the system 400 will be described below. FIG. 2 is a conceptual diagram illustrating the action example of the system 400. The system 400 illustrated in FIG. 2 includes AEDs 1A to 1D (hereinafter also referred to as “AEDs 1” generically), the portable information terminal 100, the management computer 200, and the computer apparatus 300.

A user of the system 400 is not limited particularly. However, description will be made below in a case where an administrator of a sporting event is the user of the system 400 by way of example. In the present example, the management computer 200 may be installed in an emergency head office or the like of the sporting event. In addition, the user of the portable information terminal 100 is an emergency staff placed at any place in the sporting event. The portable information terminal 100 may be a smartphone carried by the emergency staff. In addition, some emergency staffs carrying the portable type AEDs 1 may be users U of the AEDs 1. One of the users U of the AEDs 1 and the user of the portable information terminal 100 may be the same person.

In the example of FIG. 2, a user U activates the AED 1A in order to rescue a person P in need for rescue. In this state, the AED 1A sends the aforementioned wireless signal to the surroundings. The portable information terminal 100 detects the AED1 activated around the portable information terminal 100 at a set time interval.

In the example of FIG. 2, the detection interval is one minute as displayed on the display unit 140. The display unit 140 is a touch panel. By touching the display unit 140, the detection interval can be changed. Incidentally, by operating an operator on a right side of a place indicating “AED power check” in the display unit 140, the detection of the AED 1 may be suspended.

A range in which the AEDs 1 can be detected by the portable information terminal 100 is a range where a wireless signal sent from each of the AEDs 1 can reach. When the wireless signal is a radio beacon based on a wireless LAN, the range in which the wireless signal can reach is, for example, a circle with a radius of about 100 m from the AED 1. In addition, when the wireless signal is a radio beacon based on Bluetooth, the range in which the wireless signal can reach is, for example, a circle with a radius of about 10 m from the AED 1.

In the example of FIG. 2, the AED 1A is present in the range where the portable information terminal 100 can detect the wireless signal. Accordingly, the portable information terminal 100 detects the fact that the AED 1A has been activated. Incidentally, at this stage, the portable information terminal 100 only detects the wireless signal, and communication connection between the AED 1A and the portable information terminal 100 is not established. The communication connection between the AED 1A and the portable information terminal 100 may be, for example, established at a predetermined timing, such as after the AED 1A has completed a series of life rescue actions.

The AEDs 1B to 1D are also present in the range where the portable information terminal 100 can detect the wireless signal. However, since the AEDs 1B to 1D are not activated and do not send wireless signals, the AEDs 1B to 1D are not detected. In addition, assume that the portable information terminal 100 is set so as to detect only a specific AED 1 corresponding to second identification information that has been registered in the registration information 122, and the AEDs 1B to 1D are not set as detection targets. In this case, the portable information terminal 100 does not detect activation of the AEDs 1B to 1D even if the AEDs 1B to 1D have been activated.

Upon detection of the activation of the AED 1A, the portable information terminal 100 acquires position information of the portable information terminal 100 by use of the GPS sensor provided in the portable information terminal 100. In addition, the portable information terminal 100 transmits notification information including the position information to the management computer 200.

The management computer 200 receives the notification information. Upon reception of the notification information, the management computer 200 may notify a staff or the like in the emergency head office or the like of the fact that the notification information has been received. In addition, in accordance with an operation input or the like of the staff, a position 242 indicated by the position information included in the notification information, and a map 241 around the position 242 are displayed on the display unit 240. The management computer 200 transmits the notification information for notification about the activation of the AED 1A or the aforementioned position information to the computer apparatus 300 as occasion demands.

In addition, at a predetermined timing after the AED 1A has completed the series of life rescue actions, the AED 1A transmits physiological information such as electrocardiogram data of the person Pin need for rescue to the portable information terminal 100. The portable information terminal transmits the received physiological information to the management computer 200. In addition, the management computer 200 transmits the received physiological information to the computer apparatus 300 as occasion demands.

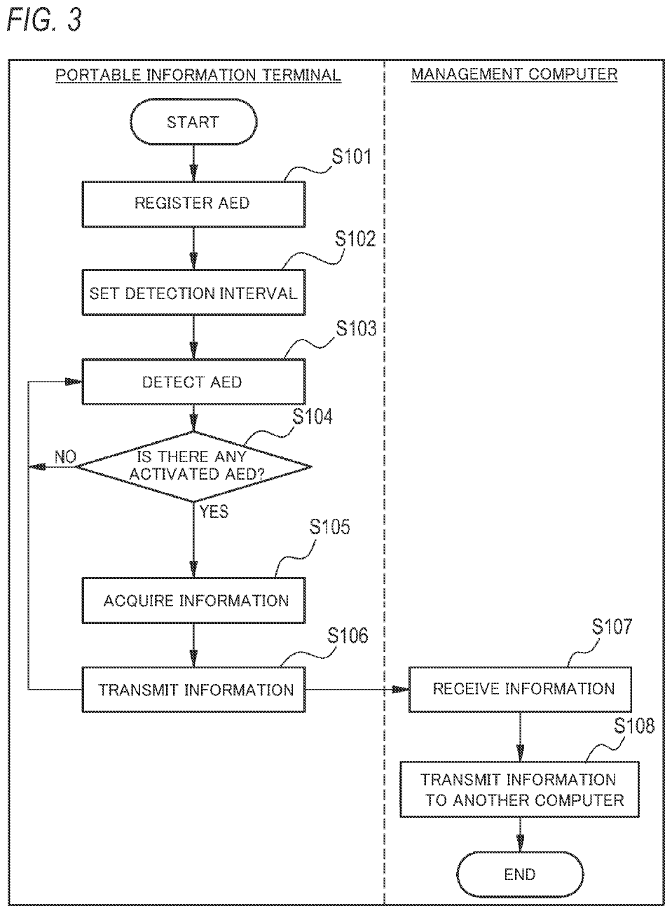

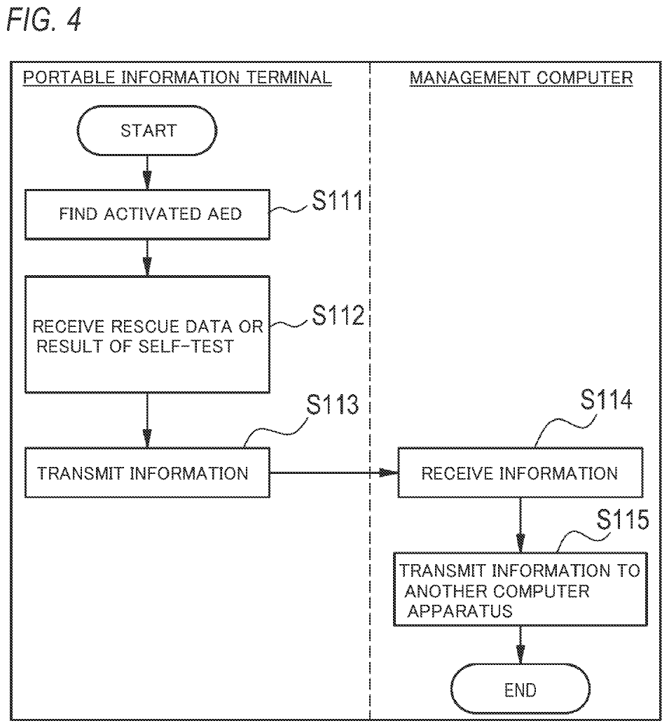

Next, action examples of the portable information terminal 100 and the management computer 200 in the system 400 will be described in detail by use of flowcharts. FIG. 3 and FIG. 4 are flowcharts for explaining examples of action processes executed in the system 400. Incidentally, various processings illustrated in FIG. 3 and FIG. 4 may be rearranged in sequence or executed in parallel without causing any contradiction.

First, the flowchart of FIG. 3 will be described. The flowchart of FIG. 3 explains an action process related to detection of an AED 1. First, the portable information terminal 100 registers, in the portable information terminal 100, second identification information of the AED 1 which is set as a detection target (step S101). In the step S101, individual information of the AED 1, individual information of the portable information terminal 100, or individual information of the user of the portable information terminal 100 may be registered in place of the second identification information or in addition to the second identification information in the portable information terminal 100. The second identification information or the individual information is stored as registration information 122 in the memory 120.

The portable information terminal 100 sets an interval for detecting a wireless signal of the AED 1 (step S102). In addition, in the step S102, a detection target or targets of the portable information terminal 100 may be set. Specifically, one or more AEDs 1 of the AEDs 1 corresponding to pieces of the second identification information or pieces of the individual information that have been registered in the registration information 122 may be set as the detection targets. Incidentally, when the detection targets are not set individually, all of the AEDs 1 which can be detected by the program 121 may be the detection targets.

The steps S101 and S102 may be executed based on an operation input of the user of the portable information terminal 100, for example, only when the user judges that the steps S101 and S102 are necessary.

The portable information terminal 100 detects presence/absence of the activated AED 1 based on presence/absence of the wireless signal sent by the activated AED 1 (step S103). When the activation of the AED 1 which is the detection target has not been detected (NO in a step S104), the step S103 is repeated at the time interval set in the step S102.

When the activation of the AED 1 which is the detection target has been detected (YES in the step S104), the portable information terminal 100 acquires predetermined information (step S105). The predetermined information acquired in the step S105 may be the position information of the portable information terminal 100, and/or the individual information etc. stored as the registration information 122. Incidentally, the step S105 is preferably executed. However, the step S105 is not essential action processing in the system 400.

The portable information terminal 100 transmits the notification information for notification about the activation of the AED 1 to the management computer (step S106). It is preferable that the position information, the individual information etc. acquired in the step S105 is included in the notification information.

Here, the communication connection between the portable information terminal 100 and the AED 1 is not established during execution of the steps S104 to S106. While the steps S104 to S106 are executed, for example, polling or the like between the portable information terminal 100 and the AED 1 is not executed but the portable information terminal 100 side only detects the wireless signal unilaterally.

The management computer 200 receives the notification information transmitted in the step S106 (step S107). Next, the management computer 200 transmits the information for notification about the activation of the AED 1 or the position information of the portable information terminal 100 to the computer apparatus 300 (step S108). Then, the management computer 200 ends the process. The step S108 may be executed based on an operation input of the user of the management computer 200, for example, only when the user judges that the step S108 is necessary.

Next, the flowchart of FIG. 4 will be described. The flowchart of FIG. 4 mainly explains an action process after an AED 1 has completed a series of rescue actions or after the AED 1 has completed a self-test.

The portable information terminal 100 establishes communication connection with the AED 1 (step S111). In the step S111, for example, authentication based on polling etc. is executed so that transmission/reception of data between the AED 1 and the portable information terminal 100 can be made.

Next, the portable information terminal 100 receives rescue data that the AED 1 has acquired by the life rescue actions from the AED 1 (step S112). The rescue data received in the step S112 are, for example, physiological information such as electrocardiogram data of a person in need for rescue. In addition, after the AED 1 has completed the self-test, the portable information terminal 100 receives a result of the self-test in the step S112.

Next, the portable information terminal 100 transmits the rescue data or the result of the self-test acquired in the step S112 to the management computer 200 (step S113). Next, the management computer 200 receives the information transmitted in the step S113 (step S114).

Next, the management computer 200 transmits the information acquired in the step S114 to the computer apparatus 300 (step S115). Incidentally, when the information acquired in the step S114 is the result of the self-test, the step S115 does not have to be executed. In addition, the step S115 may be executed based on an operation input of the user, for example, only when the user of the management computer 200 judges that the step 115 is necessary.

Successively, effects obtained by the system 400 according to the present embodiment or by the respective constituents included in the system 400 will be described.

The portable information terminal 100 detects the wireless signal from the AED 1. Upon the detection, the portable information terminal 100 notifies the management computer 200 of the activation of the AED 1 without establishing communication connection with the activated AED 1. That is, the portable information terminal 100 can more surely contact the manager or the like about the fact that the AED 1 has been activated while reducing a load on the AED 1 due to avoidance of the communication connection between the activated AED 1 and the portable information terminal 100.

In addition, an approximate position at which the AED 1 is activated can be confirmed based on the position information of the portable information terminal 100. Accordingly, life rescue activity can be supported easily on the side of the user of the management computer 200 or the user of the computer apparatus 300.

In addition, the configuration is made not to acquire the position information of the AEDs 1 but to acquire the position information of the portable information terminal 100. Accordingly, it is not necessary to mount the GPS sensor on each of the AEDs 1 so that battery consumption or manufacturing cost of the AED 1 side can be suppressed. In addition, when the portable information terminal 100 supports acquisition of the position information corresponding to its usage area, nothing is required to be additionally mounted on the AED 1 side about the position information. That is, even if the AEDs 1 are of a single model type, the position information corresponding to various areas can be acquired.

In addition, the portable information terminal 100 transmits the individual information to the management computer 200. Accordingly, the individual information about the AED 1, the portable information terminal 100 or the user of the portable information terminal 100 can be known on the user side of the management computer 200. As a result, the user of the management computer 200 can easily support the life rescue activity. For example, when a contact address such as a telephone number of the portable information terminal 100 is included in the individual information, the user of the management computer 200 can also contact the user of the portable information terminal 100 to give a proper instruction thereto.

In addition, at the predetermined timing, such as after the series of life rescue actions has been terminated, the AED 1 transmits, to the portable information terminal, the physiological information such as the electrocardiogram data of the person in need for rescue and the portable information terminal 100 transmits the physiological information received from the AED 1 to the management computer 200. As a result, it is possible to determine whether a further emergency measure on the person in need for rescue is required or not so that the user side of the management computer 200 can easily support the rescue activity. In addition, the user of the management computer 200 can transmit the physiological information to the computer apparatus 300 installed in the hospital or the like as occasion demands. Accordingly, a suitable and rapid measure can be easily made in the hospital or the like.

In addition, the portable information terminal 100 can set only a specific AED 1 as the detection target. Accordingly, it is possible to prevent such a situation that the user of the management computer 200 is confused because the user side is also notified of activation from any other AED 1 not managed by the user. In addition, the detection target is set suitably. Thus, when the user or the like of the management computer 200 organizes emergency staffs to build a rescue system, the rescue system can be built easily and suitably.

In addition, the time interval at which the AED 1 is detected can be set by the user side of the portable information terminal 100. Thus, for example, it is possible to select the detection interval suitably in accordance with the scale of the rescue system, the role of the user, a remaining level of the battery of the portable information terminal 100, etc.

In addition, when the wireless communicator 90 of the AED 1 is compatible with the wireless LAN standard or the Bluetooth standard, a beacon conforming to these standards is sent as the wireless signal. Accordingly, the AED 1 is easily detected. In addition, when the wireless communicator 90 is compatible with the wireless LAN standard, the AED 1 can be detected within a comparatively wide range of a circle with a radius of about 100 m from the portable information terminal 100. On the other hand, when the wireless communicator 90 is compatible with the Bluetooth standard, a circle with a radius of about 10 m from the portable information terminal 100 is within the detection range. Accordingly, a difference between the position of the AED 1 and the position indicated by the position information of the portable information terminal 100 is reduced so that the position where the AED 1 is activated can be known more accurately.

The aforementioned embodiment is merely exemplar in order to make the presently disclosed subject matter easy to understand. The configuration according to the aforementioned embodiment can be changed/improved properly without departing from the gist of the presently disclosed subject matter. The present application is based on Japanese Patent Application No. 2020-098726 filed on Jun. 5, 2020, the entire contents of which are hereby incorporated by reference.

### REFERENCE SIGNS LIST

1: automated external defibrillator (AED)
10: controller (wireless communication module)
100: portable information terminal
110: controller (processor)
120: memory
121: program
200: management computer
300: computer apparatus
400: system

1. A non-transitory computer-readable medium having recorded thereon a program that makes a processor of a portable information terminal execute:
a detection step of detecting presence/absence of an automated external defibrillator in an activated state based on presence/absence of a wireless signal sent from the automated external defibrillator, the wireless signal becoming detectable in response to activation of the automated external defibrillator having a wireless communication module; and
a first transmission step of transmitting notification information for notification about the activation of the automated external defibrillator to a management computer in response to the automated external defibrillator in the activated state being detected in the detection step.

2. The non-transitory computer-readable medium according to claim 1, making the processor further execute a step of acquiring position information of the portable information terminal in response to the automated external defibrillator in the activated state being detected in the detection step, wherein
the notification information includes the position information.

3. The non-transitory computer-readable medium according to claim 1, making the processor further execute a step of storing, in a memory of the portable information terminal, individual information about the portable information terminal, the automated external defibrillator or a user of the portable information terminal, wherein
the notification information includes the individual information.

4. The non-transitory computer-readable medium according to claim 1, making the processor further execute:
a step of receiving at least a portion of physiological information of a person in need for rescue acquired by the automated external defibrillator from the automated external defibrillator; and
a second transmission step of transmitting the portion of the physiological information to the management computer.

5. The non-transitory computer-readable medium according to claim 1, making the processor further execute a step of storing, in a memory of the portable information terminal, information about the automated external defibrillator set as a detection target, wherein
in the detection step, presence/absence of the automated external defibrillator whose information has been stored in the memory and which is in the activated state is detected.

6. The on-transitory computer-readable medium according to claim 1, wherein
the detection step is executed at a predetermined interval set by a user of the portable information terminal.

7. The non-transitory computer-readable medium according to claim 1, wherein
the wireless communication module is a wireless LAN module or a Bluetooth (registered trademark) module.

8. A portable information terminal comprising:
a detector that detects presence/absence of an automated external defibrillator in an activated state based on presence/absence of a wireless signal sent from the automated external defibrillator, the wireless signal becoming detectable in response to activation of the automated external defibrillator having a wireless communication module; and
a first transmitter that transmits notification information for notification about the activation of the automated external defibrillator to a management computer when the automated external defibrillator in the activated state has been detected.

9. A system that is realized by one or more portable information terminals, and a management computer capable of making communication connection with the portable information terminals, wherein:
each of the portable information terminals includes:
a detector that detects presence/absence of an automated external defibrillator in an activated state based on presence/absence of a wireless signal sent from the automated external defibrillator, the wireless signal becoming detectable in response to activation of the automated external defibrillator having a wireless communication module, and
a first transmitter that transmits notification information for notification about the activation of the automated external defibrillator to the management computer when the automated external defibrillator in the activated state has been detected; and
the management computer includes:
a receiver that receives the notification information transmitted by the first transmitter, and
a third transmitter that transmits predetermined information to an arbitrary computer apparatus when the notification information has been received.

10. The system according to claim 9, wherein:
the portable information terminal further includes an acquirer that acquires position information of the portable information terminal when the automated external defibrillator in the activated state has been detected;
the position information is included in the notification information; and
the position information is included in the predetermined information.

11. A method for notification about activation of an automated external defibrillator, which is executed in a portable information terminal including a processor so that the method makes the processor execute:
a detection step of detecting presence/absence of the automated external defibrillator in an activated state based on presence/absence of a wireless signal sent from the automated external defibrillator, which wireless signal can be detected according to the activation of the automated external defibrillator having a wireless communication module; and
a first transmission step of transmitting notification information for notification about the activation of the automated external defibrillator to a management computer when the automated external defibrillator in the activated state has been detected.
